(19) United States
(12) Patent Application Publication
(10) Pub. No.: US 2005/0234506 A1
(43) Pub. Date: Oct. 20, 2005

(54) Blepharitz eyelid compressor

(76) Inventor: Rubin G. Weser, Whitestone, NY (US)

Correspondence Address: EDWIN TARVER, 20121 VENTURA BOULEVARD, SUITE 302, WOODLAND HILLS, CA, 91364, US

(21) Appl. No.: 11/101,968

(22) Filed: Apr. 8, 2005

Related U.S. Application Data
(60) Provisional application No. 60/562,512, filed on Apr. 15, 2004.

(51) Int. Cl. A61B 17/28

(52) U.S. Cl. 606/205

(57) ABSTRACT

The present invention relates to a scissor like surgical instrument, operating as an eyelid compressor, used by ophthalmologists to extract oil from the eyelid glands to reduce inflammation, redness, crusting, and swelling of the eyelids caused by blepharitis. The eyelid compressor comprises of a pair of elongated members that are pivotally connected at an intermediate point and are pressed against each other in the transverse direction to perform the squeezing action. A pair of sterile pad is fastened to inner faces of the elongated members and they are of disposable nature.

### CROSS-REFERENCE TO RELATED APPPLICATION

This application claims the benefit of Provisional application Ser. No. 60/562,512, filed Apr. 15, 2004.

### FEDERALLY SPONSORED RESEARCH

Not Applicable

### SEQUENCE LISTING OR PROGRAM

Not Applicable

### BACKGROUND

The present invention relates generally to surgical instruments and more particularly to surgical instruments used by ophthalmologists to extract the oil from the eyelid glands.

Blepharitis is a chronic inflammation of the eyelids and is one of the most common disorders of the eye. It usually results from a dysfunction of the small oil glands of the eyelids. When these glands are not secreting the oils involved in tear formation, blepharitis can result.

As a part of the ageing process, the skin of the eyelid can stretch, the muscles weaken and fatty deposits accumulate around their eyelids. When this happens, oils and other secretions may build up on the eyelid surface and eyelashes, resulting in a crusting and flaking material that can enter the eye. Swelling or inflammation of the eyelids is also common. Other symptoms can include a sandy, gritty sensation, with burning, itching and/or redness.

Although Blepharitis is a chronic problem, applying warm compresses and eyelid scrubs can help control it. In some cases, it may be necessary to prescribe an antibiotic ointment or pills. A person can also undergo Blepharoplasty, a corrective eyelid surgery that helps to remove excess tissue and fatty deposits around the eyes. Typically, however, ophthalmologists simply use either their hands or Q-tips to press against the eyelids and excrete the oils that cause blepharitis.

Although surgical instruments and apparatuses relating to the eye and optometry have been described in the prior art, few relate to surgical instruments or tools that can be used to extract secretions from the eyelids. Japanese Pat. No. 2002000350 to Aisaka Kenji, discloses a tool composed of a soft material with elasticity and slide-stopper on a cylindrical top. By pressing it around an eye while rotating it inwardly and downwardly, specifically in the direction form inner corner to outer corner, secretions on the upper and lower lids can be removed.

However, this tool is structurally different from the eyelid compressor of the present invention. The embodiment of the present invention offers more convenience in extracting oil from the eyelid glands. Therefore, it is a principle object of the present invention to overcome the problems and deficiencies in the art.

It is an object of the present invention to provide a scissor like surgical instrument to be used by ophthalmologists to extract oils, secretions and other substances from the eyelid glands, thereby reducing inflammation, redness, crusting, and swelling of the eyelids. It is another object of the present invention to provide a surgical instrument containing a mechanism to insert and remove sterile pads. The used pads can be discarded after each use. These and other objects will become apparent from the following accompanying drawings and description.

### SUMMARY

The present invention relates generally to surgical instruments and more particularly to a surgical instrument used by ophthalmologists to extract the oil from the eyelid glands. The surgical instrument of the present invention is in the form of a handheld mechanism with two mirror-opposed, hand-actuated handles extending from a dovetail locking mechanism through opposing arcs, and meeting again at a hinge.

Extending from the hinge are two members terminating in an arced gripping means. Two heads, arced in a concave manner in a plane perpendicular to the instrument accommodate removable surgical pads designed to press down against the eyelids and press the eyelids together.

As the eyelids are pressed together, oils and other secretions that have formed a crust on the inner and bottom surfaces of the eyelids are removed, permitting normal tear formation and proper ocular lubrication to alleviate the symptoms of blepharitis.

### BRIEF DESCRIPTION OF THE FIGURES

FIG. 1 is a side view of the eyelid compressor of the present invention, comprising the handles, hinge mechanism and pressure mechanism with pads.

FIG. 2 is a top view of the eyelid compressor of the present invention, illustrating the handles with finger grips, and pressure mechanism.

FIG. 3 is a front view of the eyelid compressor of the present invention, illustrating the hinge mechanism and pressure mechanism with removable pads, and representing an alternative embodiment of the attachment of the pressure mechanism

### FIGURES—REFERENCE NUMERALS

10 . . . Eyelid Compressor
11 . . . Elongated Member
11A . . . Anterior End of Elongated Member
12 . . . Gripping Head
13 . . . Arced Members
14 . . . Sterile Pads
15 . . . Intermediate Point
16 . . . Posterior End
17 . . . Bolt
18 . . . Gripping Surface
19 . . . Openings

### DESCRIPTION

A preferred embodiment of the present invention relates to a scissor like surgical instrument used by ophthalmologists to extract oil from the eyelid glands to reduces inflammation, redness, crusting, and swelling of the eyelids. The invention is also a reusable surgical instrument meant for treating patients suffering from Blepharitis wherein disposable sterile pads are used, and the instrument is autoclave safe.

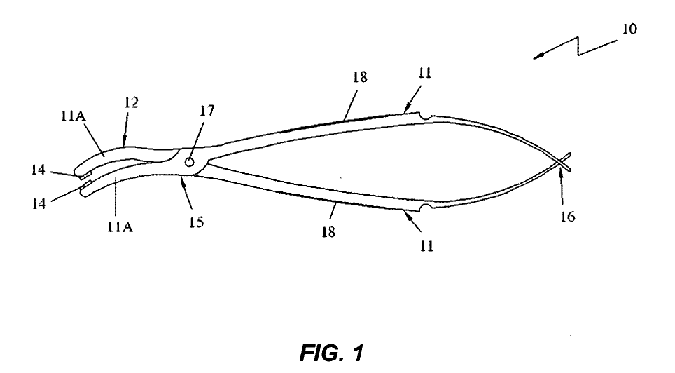

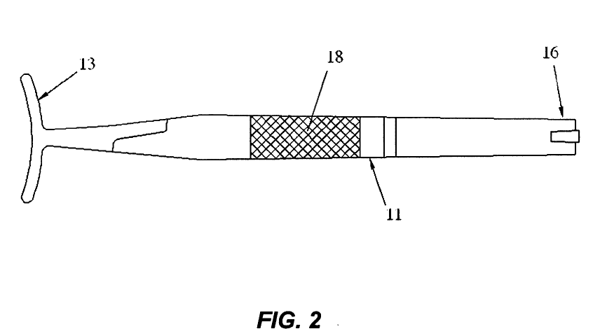

One preferred embodiment of the present invention is illustrated as shown in FIGS. 1, 2 and 3. The present invention relates to a surgical instrument that operates as an eyelid compressor 10, used by ophthalmologists to extract oil from eyelid glands, and alleviate symptoms caused by blepharitis. The eyelid compressor 10 is in the form of a two-handled scissoring mechanism associated with two arced handles or gripping members 11.

The gripping members terminate at the anterior end 11A of the compressor 10 in a gripping head 12 comprising the anterior portions of the gripping members 11A and two centrally mounted outwardly concave elongated arced members 13. Each arced member 13 accommodates one soft sterile pad 14 comprising cotton, or other similar soft absorbent material.

The scissoring movement of the elongated members 11 causes the sterile pads 14 to press against and down along the eyelids, exerting a uniform gentle pressure to help excrete oils and other material. The compressor 10 can be used to help people suffering from Blepharitis. It reduces inflammation, redness, crusting, and swelling of the eyelids.

As seen in FIG. 1, the eyelid compressor 10 comprises of a pair of elongated members 11, each of which has an application end located at the anterior end 11A at the gripping head 12 of the compressor, and a handle end that continues in the arc of the elongated member 11, meeting and interlocking at the posterior end 16 to limit the range of movement of the compressor 10. The elongated members 11 are pivotally connected at an intermediate point 15 by a bolt 17, screw, rivet, or other means. The elongated members also have a gripping surface 18 to prevent slippage when the compressor 10 is in operation.

The elongated members 11 are attached in a manner so that the respective application ends of the compressor 10 at the gripping head 12 of the compressor 10 scissor back and forth performing a compressing action against the top and bottom of the eyelids simultaneously by relative movement of the elongated members 11. The elongated members 11 are pressed against each other in the transverse direction to perform the squeezing action.

A pair of sterile pads 14 is fastened to inner faces of the anterior ends of the elongated members 11A. The pads are removable and disposable and are discarded after each use.

In one preferred embodiment of the invention, the preferred length of the eyelid compressor 10 is approximately 4.75 inches and its width is approximately 0.310 inches. The lateral length of the arced members 13 is approximately 0.75 inches. The eyelid compressor 10 is preferably made of stainless steel or titanium and can also be made of any other material having similar characteristics or properties.

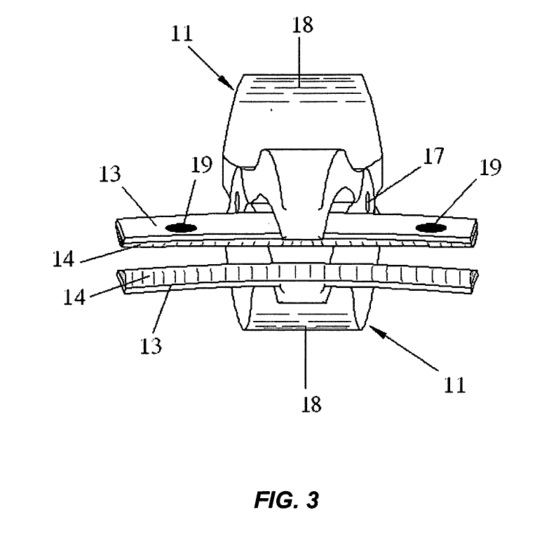

Referring to FIG. 3, openings 19 are provided on the arced members 13 of on the gripping head 12 to accommodate a pair of sterile pads 14. The eyelid compressor 10 extracts the oil from the eyelid glands by pressing the moveable part sterile pads 14 against the top and bottom of the eyelids at the same time and depressing the elongated members 11.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

While specific apparatus has been disclosed in the preceding description, it should be understood that these specifics have been given for the purpose of disclosing the principles of the present invention and that many variations thereof will become apparent to those who are versed in the art. Therefore, the scope of the present invention is to be determined by the appended claims.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

1. A scissor-like surgical instrument, which operates as an eyelid compressor, comprising: a pair of elongated members of essentially the same length, each having an application end and a handle end, said application ends having opposed inner faces and distal outer faces with arced concave outwardly facing members matching the curvature of the eyelid, said pair of elongated members being pivotally connected at an intermediate point and arranged so that the application ends together provide a surgical compressing function upon relative movement of said handle ends; and a pair of sterile pads which are attached to said inner faces of the arced members to rub along the surface of the eyelid.

2. The eyelid compressor of claim 1, wherein said instrument is made of stainless steel, titanium or any other resilient autoclave safe material.

3. The eyelid compressor of claim 2, whereby said sterile pads are composed of cotton or another soft absorbent sterile material and are disposable by nature.

4. The eyelid compressor of claim 1, wherein the point at which the pair of elongated members are pivotally connected is disposed near the application end and provides leverage for the arced members.

5. The eyelid compressor of claim 1, wherein the sterile pads are adhesively attached to the inner faces of the arced members, or are attached by pilot holes or another temporarily adhering means.

6. The eyelid compressor of claim 1, wherein the elongated members terminate at the posterior end of the compressor at an interlocking point to limit the range of movement of the anterior end and associated sterile pads.

7. The eyelid compressor of claim 1, wherein the elongated members serve as handles and are equipped with a textured gripping surface to assist in the operation of the compressor

8. A method of alleviating the symptoms of blepharitis, or other similar conditions by pulling apart the elongated members of the present invention, applying the pads adhered to the arced members to the outer surface of the upper and lower eyelids, and applying a closing pressure on the elongated members of the handle end of the apparatus, thereby pushing the sterile pads upwardly and downwardly along the surface of the eyelids, and the eyelids closed and firmly together to remove oils and other material.
